(19) United States
(12) Patent Application Publication
(10) Pub. No.: US 2025/0235663 A1
(43) Pub. Date: Jul. 24, 2025

(54) CATHETER AND CATHETER SYSTEM

(71) Applicant: TERUMO KABUSHIKI KAISHA, Tokyo (JP)

(72) Inventor: Takashi ITO, Shizuoka-shi (JP)

(73) Assignee: TERUMO KABUSHIKI KAISHA, Tokyo (JP)

(21) Appl. No.: 19/174,542

(22) Filed: Apr. 9, 2025

Related U.S. Application Data
(63) Continuation of application No. PCT/JP2023/030567, filed on Aug. 24, 2023.

(30) Foreign Application Priority Data
Oct. 11, 2022 (JP) 2022-163443

(51) Int. Cl. A61M 25/00; A61B 17/00; A61B 17/22; A61B 90/00; A61M 25/01

(52) U.S. Cl. CPC A61M 25/0068; A61B 17/00234; A61B 17/22; A61B 90/39; A61M 25/0023; A61M 25/0108; A61B 2017/00238; A61B 2017/00305; A61B 2017/22094; A61B 2090/3966; A61M 2205/32

(57) ABSTRACT

An antegrade catheter of a catheter system includes a tubular first marker disposed at a distal end part of a first catheter body. The first marker is formed in a reverse tapered shape expanding in diameter toward the distal direction of the first catheter body. An axial distance between the most distal end of the first marker and the most distal end of the first catheter body in the axial direction of the first catheter body is 0.5 mm or less.

### CROSS-REFERENCES TO RELATED APPLICATIONS

This application is a continuation of International Application No. PCT/JP2023/030567 filed on Aug. 24, 2023, which claims priority to Japanese Patent Application No. 2022-163443 filed on Oct. 11, 2022, the entire content of both of which is incorporated herein by reference.

### TECHNOLOGICAL FIELD

The present invention generally relates to a catheter and a catheter system that are inserted into a lumen of a living body.

### BACKGROUND DISCUSSION

JP 2010-279546 A discloses a catheter system including a radiopaque marker at a distal end part. The catheter system includes a catheter body having flexibility and a radiopaque marker embedded in a distal end part of the catheter body. The radiopaque marker is a cylindrical body formed of a metal material.

When there is a lesion (stenosis) in the lumen of a patient, the distal end of the catheter is moved forward along the lumen under X-ray contrast effect. The position of the distal end of the catheter is confirmed by the radiopaque marker, and the distal end of the catheter is delivered to the lesion for treatment.

### SUMMARY

When lower limb blood vessel treatment is performed on a lesion of a lower limb artery of a patient, for example, an antegrade catheter is inserted into a blood vessel in advance in an antegrade approach, and then another retrograde catheter is inserted into the blood vessel in a retrograde approach from a direction opposite to the antegrade catheter. By inserting the distal end of another retrograde catheter into the distal end of the antegrade catheter in the vicinity of the lesion, the distal ends of the antegrade catheter and the retrograde catheter are arranged at a predetermined position in the vicinity of the lesion, and the balloon catheter is delivered to the lesion through the antegrade catheter.

In the catheter of JP 2010-279546 A, since the radiopaque marker is disposed at a position away from the distal end of the catheter body, in a case where the catheter of JP 2010-279546 A is used as an antegrade catheter and a retrograde catheter, it is difficult to align and insert the distal end of the retrograde approach with the distal end of the catheter inserted by the antegrade approach under angiography.
(1) A first aspect disclosed here involves a catheter that is inserted into a lumen of a living body and moves forward along the lumen, the catheter comprising: a tubular catheter body having a lumen; and a tubular marker containing a radiopaque material and disposed at a distal end part of the catheter body, wherein the marker is formed in a reverse tapered shape expanding in diameter toward a distal direction of the catheter body, and a distance between a most distal end of the marker and a most distal end of the catheter body in an axial direction of the catheter body is 0.5 mm or less.

According to this catheter, since the axial distance between the most distal end of the marker arranged at the distal end part of the catheter and the most distal end of the catheter body is 0.5 mm or less, the distal position of the catheter can be accurately confirmed under angiography when the catheter is moved forward along the lumen of the living body. Since the marker is formed in the reverse tapered shape, it is easy to identify the marker based on the shape that can be confirmed under angiography, and the marker is prevented from falling off from the catheter body.
(2) In the catheter according to (1), the catheter may be an antegrade catheter that moves forward to a peripheral side of the living body along the lumen or a retrograde catheter that moves forward to a central side of the living body along the lumen and has a distal end insertable into a distal end of the antegrade catheter.

With this configuration, by providing the marker at the distal end of the antegrade catheter or the distal end of the retrograde catheter, it is easy to align the distal end of the antegrade catheter with the distal end of the retrograde catheter.
(3) In the catheter according to (1), the catheter may be an antegrade catheter that moves forward to a peripheral side of the living body along the lumen.
(4) In the catheter according to (3), the distal end part of the catheter body may have an inner surface portion that is a part of the lumen and has a reverse tapered shape expanding in diameter in the distal direction, and the marker may be disposed along the inner surface portion.

With this configuration, when the distal end of the retrograde catheter is inserted into the lumen at the distal end of the antegrade catheter, the insertability of the distal end of the antegrade catheter and the distal end of the retrograde catheter with each other can be enhanced by the inner surface portion of the distal end part of the catheter body.
(5) In the catheter according to (1), the catheter may be a retrograde catheter that moves forward to a central side of the living body along the lumen, a distal end of the retrograde catheter may be insertable into the lumen at a distal end of an antegrade catheter moving forward to a peripheral side of the living body along the lumen, and an outer peripheral surface of the distal end of the retrograde catheter may have a tapered outer surface portion whose diameter decreases in the distal direction.

With this configuration, when the distal end of the retrograde catheter is inserted into the lumen at the distal end of the antegrade catheter, the insertability of the retrograde catheter with respect to the antegrade catheter can be enhanced by the outer surface portion.
(6) A second aspect involves a catheter system including a catheter that is inserted into a lumen of a living body and moves forward along the lumen. The catheter system includes: an antegrade catheter that moves forward to a peripheral side of the living body along the lumen; and a retrograde catheter that moves forward to a central side of the living body along the lumen. The antegrade catheter includes: a tubular first catheter body having a first lumen; and a tubular first marker containing a radiopaque material and disposed at a distal end part of the first catheter body. The first marker is formed in a reverse tapered shape expanding in diameter in a distal direction of the first catheter body. A distance between a most distal end of the first marker and a most distal end of the first catheter body in an axial direction of the first catheter body is 0.5 mm or less. A distal end of the retrograde catheter is insertable into a distal end of the antegrade catheter. The retrograde catheter includes: a tubular second catheter body having a second lumen; and a tubular second marker containing a radiopaque material and disposed at a distal end part of the second catheter body. The second marker is formed in a reverse tapered shape expanding in diameter in a distal direction of the second catheter body. A most distal end of the second marker is disposed on a proximal side with respect to a most distal end of the second catheter body. A distance between the most distal end of the second marker and the most distal end of the second catheter body in an axial direction of the second catheter body is 0.5 mm or less.
(7) A third aspect involves a treatment method for treating a lesion in a lumen of a living body using an antegrade catheter that moves forward to a peripheral side of the living body along the lumen of the living body and a retrograde catheter that moves forward to a central side of the living body along the lumen. The antegrade catheter includes: a tubular first catheter body having a first lumen; and a tubular first marker having radiopacity and disposed at a distal end part of the first catheter body. The first marker is formed in a reverse tapered shape expanding in diameter in a distal direction of the first catheter body. A distance between a most distal end of the first marker and a most distal end of the first catheter body in an axial direction of the first catheter body is 0.5 mm or less. The retrograde catheter includes: a tubular second catheter body having a second lumen; and a tubular second marker containing a radiopaque material and disposed at a distal end part of the second catheter body. The second marker is formed in a reverse tapered shape expanding in diameter in a distal direction of the second catheter body. A most distal end of the second marker is disposed on a proximal side with respect to a most distal end of the second catheter body. A distance between the most distal end of the second marker and the most distal end of the second catheter body in an axial direction of the second catheter body is 0.5 mm or less. In the lumen of the living body, when the antegrade catheter moves forward to the peripheral side and the retrograde catheter moves forward to the central side, by aligning the first marker and the second marker while confirming positions of the first marker and the second marker under angiography, the distal end part of the antegrade catheter and the distal end part of the retrograde catheter are aligned, and a distal end of the retrograde catheter is inserted into a distal end of the antegrade catheter.

According to an aspect of the disclosure here, a marker is disposed at a distal end part of a catheter, and an axial distance between a most distal end of the marker and a most distal end of a catheter body is 0.5 mm or less. As a result, when the catheter is moved forward along the lumen of the living body, the distal position of the catheter can be accurately confirmed under angiography. Since the marker is formed in the reverse tapered shape, the marker can be easily identified based on a shape that can be confirmed under angiography, and the marker is prevented from falling off from the catheter body.

### BRIEF DESCRIPTION OF THE DRAWINGS

FIG. 1 is a diagram illustrating an overall configuration of a catheter system according to one embodiment disclosed as an example.

FIG. 2 is an enlarged cross-sectional view illustrating a distal end part of an antegrade catheter.

FIG. 3 is a cross-sectional view taken along the section line III-III in FIG. 2.

FIG. 4 is an enlarged cross-sectional view illustrating a distal end part of the retrograde catheter.

FIG. 5 is a cross-sectional view taken along the section line V-V in FIG. 4.

FIG. 6 is an explanatory view illustrating an initial state when treatment is performed using the catheter system.

FIG. 7 is an explanatory view illustrating a state in which the distal end part of the antegrade catheter is delivered to the upstream end portion of a lesion (CTO).

FIG. 8 is an explanatory view illustrating a state in which the distal end part of the retrograde catheter is inserted into the distal end part of the antegrade catheter in a lesion (CTO).

### DETAILED DESCRIPTION

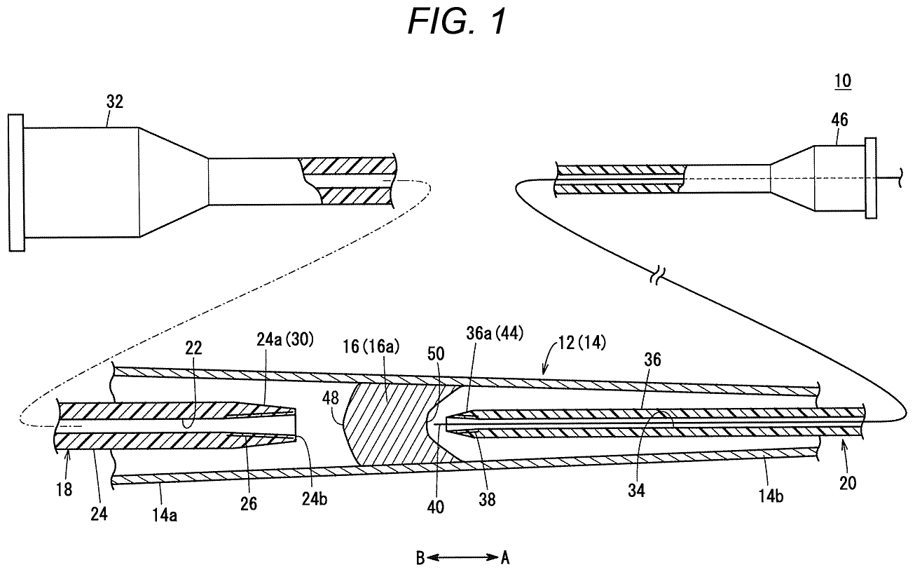

As illustrated in FIG. 1, a catheter system 10 according to the present embodiment is used, for example, for treatment of a lesion 16 (a stenosis, a blocked portion, or the like) generated in a blood vessel 14 of a living body 12. Specifically, the catheter system 10 is used for lower limb blood vessel treatment in which CTO 16a (chronic total occlusion, lesion) generated in the blood vessel 14 of the lower limb of the living body 12 is treated by an antegrade approach and a retrograde approach. The catheter system 10 may also be used to treat a lumen other than the blood vessel 14, for example, the lesion 16 in a living organ such as a bile duct, a trachea, an esophagus, a urethra, or other organs.

The catheter system 10 is inserted into the blood vessel 14 of the living body 12 and can progress along the blood vessel 14. The catheter system 10 includes an antegrade catheter 18 used for an antegrade approach in lower limb blood vessel treatment and another retrograde catheter 20 used for a retrograde approach in lower limb blood vessel treatment.

The antegrade catheter 18 is a catheter that moves forward to the peripheral side (ankle side, direction of arrow A) of the living body 12 along the blood vessel 14 of the living body 12 in lower limb blood vessel treatment.

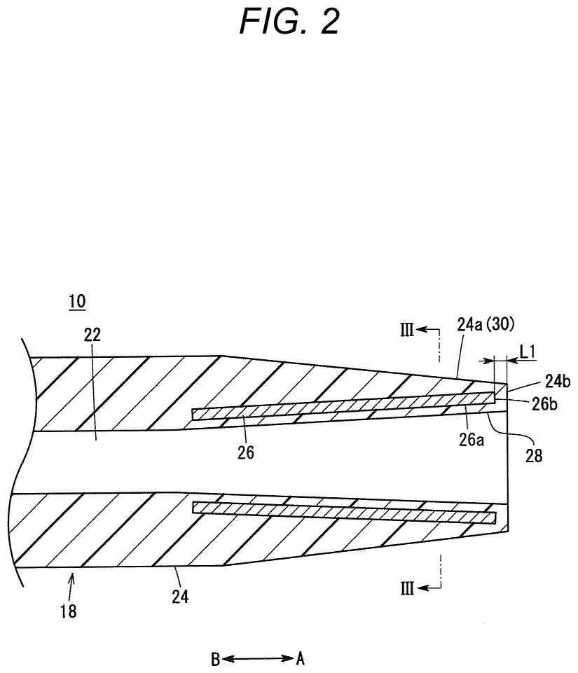

As illustrated in FIG. 2, the antegrade catheter 18 includes a tubular first catheter body 24 having a first lumen 22 and a first marker 26 disposed at a distal end part 24a of the first catheter body 24.

The first catheter body 24 is formed of a flexible resin material. Specifically, a constituent material from which the first catheter body 24 may be fabricated includes resin materials having a certain degree of flexibility such as polyolefins such as polyethylene, polypropylene, and ethylene-propylene copolymers, polyesters such as polyethylene terephthalate and polybutylene terephthalate, and various elastomers such as polystyrene, polyvinyl chloride, polyurethane, polyamide, or polyolefin elastomer, polyester elastomer, polyurethane elastomer, and polyamide elastomer, and the resin materials may be blended, laminated, or arranged in multiple stages in the axial direction, or a reinforcing body may be arranged. The first lumen 22 is disposed inside the first catheter body 24. The first lumen 22 extends along the first catheter body 24. Since the antegrade catheter 18 is used for treatment of the CTO 16a, the distal end part 24a of the first catheter body 24 is not made of a soft material such as a rubber material (elastomer material), and has hardness suitable for treatment of the CTO 16a.

The distal end part 24a of the first catheter body 24 includes a first inner surface portion 28 and a first outer surface portion 30. The first inner surface portion 28 is a part of the first lumen 22 and has a reverse tapered shape expanding in diameter in the distal direction (direction of arrow A). In other words, the first inner surface portion 28 has a tapered shape in which the diameter gradually decreases from a most distal end 24b of the first catheter body 24 toward the proximal end. The distal end part 24a of the first catheter body 24 is an end portion in the advancing direction when the antegrade catheter 18 is inserted into the blood vessel 14 and moved forward. The first inner surface portion 28 is disposed in a predetermined range from the most distal end 24b of the first catheter body 24 toward the proximal end.

The first outer surface portion 30 is disposed on the outer peripheral surface of the first catheter body 24. The first outer surface portion 30 has a tapered shape whose diameter decreases in the distal direction (direction of arrow A) of the first catheter body 24. The first outer surface portion 30 is disposed in a predetermined range from the most distal end 24b of the first catheter body 24 toward the proximal end. That is, the distal end part 24a of the first catheter body 24 gradually tapers toward the most distal end 24b (distal direction, direction of arrow A).

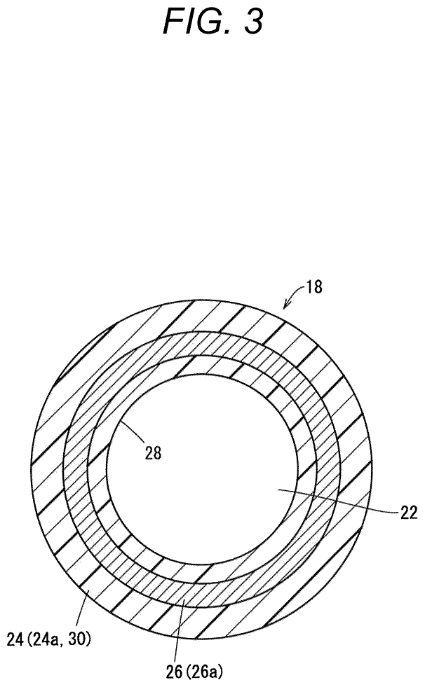

As illustrated in FIG. 2, the first marker 26 is formed in a tubular shape (see FIG. 3) from a radiopaque material (for example, gold, platinum, tungsten, a mixture thereof, or the like). The first marker 26 enables the distal position (most distal end 24b) of the antegrade catheter 18 to be visually recognized under angiography in the living body 12.

The first marker 26 is embedded in the distal end part 24a of the first catheter body 24. The first marker 26 is formed in a reverse tapered shape expanding in diameter toward the distal direction (direction of arrow A) of the first catheter body 24. The first marker 26 is disposed along the first inner surface portion 28 of the first catheter body 24. The first inner surface portion 28 of the first catheter body 24 and the first marker 26 are substantially parallel. A part of the first marker 26 may be exposed to the first inner surface portion 28. Since the first marker 26 has a reverse tapered shape, the first marker 26 is prevented from falling off from the distal end part 24a of the first catheter body 24 in the distal direction (direction of arrow A).

A distal end part 26a of the first marker 26 has a most distal end 26b disposed in the most distal direction (direction of arrow A). The most distal end 26b of the first marker 26 is disposed on the proximal side (direction of arrow B) of the most distal end 24b of the first catheter body 24. In the axial direction (directions of arrows A and B) of the first catheter body 24, an axial distance L1 between the most distal end 26b of the first marker 26 and the most distal end 24b of the first catheter body 24 is 0.5 mm or less (greater than zero). That is, the most distal end 26b of the first marker 26 is disposed within 0.5 mm on the proximal side (direction of arrow B) from the most distal end 24b of the first catheter body 24. The most distal end 26b of the first marker 26 is not exposed to the outside from the most distal end 24b of the first catheter body 24. The axial length of the first marker 26 is, for example, about 0.5 mm to 1.0 mm along the extending direction of the first catheter body 24.

As illustrated in FIG. 1, the proximal end of the first catheter body 24 includes a first hub 32. The first hub 32 has a tubular shape. The proximal end of the first hub 32 is open.

The retrograde catheter 20 moves forward to the central side (heart side, direction of arrow B) of the living body 12 along the blood vessel 14 of the living body 12 in the lower limb blood vessel treatment. When the retrograde catheter 20 moves forward toward the central side, a distal end part 36a of the retrograde catheter 20 can be inserted into the distal end part 24a of the antegrade catheter 18.

The retrograde catheter 20 includes a tubular second catheter body 36 having a second lumen 34 and a second marker 38 disposed at the distal end part 36a of the second catheter body 36.

The second catheter body 36 is formed of a flexible resin material. Specifically, as a constituent material from which the second catheter body 36 may be fabricated, resin materials having a certain degree of flexibility such as polyolefins such as polyethylene, polypropylene, and ethylene-propylene copolymers, polyesters such as polyethylene terephthalate and polybutylene terephthalate, and various elastomers such as polystyrene, polyvinyl chloride, polyurethane, polyamide, or polyolefin elastomer, polyester elastomer, polyurethane elastomer, and polyamide elastomer are used, and the resin materials may be blended, laminated, or arranged in multiple stages in the axial direction, or a reinforcing body may be arranged. The diameter of the second catheter body 36 is smaller than the diameter of the first catheter body 24. The second lumen 34 is disposed inside the second catheter body 36. The second lumen 34 extends along the second catheter body 36. The second lumen 34 is a passage through which a guide wire 40 can be inserted. Since the retrograde catheter 20 is used for treatment of the CTO 16a, the distal end part 36a of the second catheter body 36 is not made of a soft material such as a rubber material (elastomer material), and has hardness suitable for treatment of the CTO 16a.

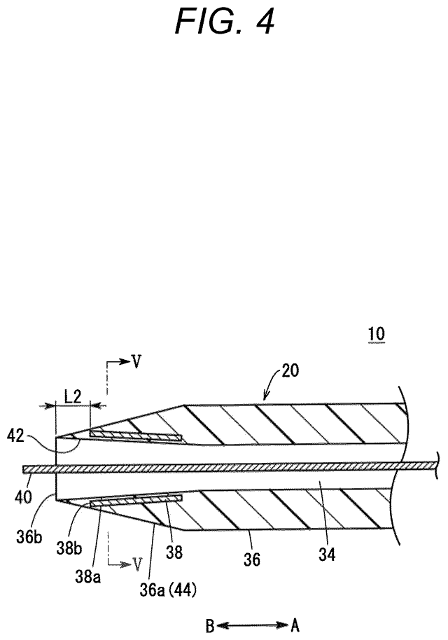

As illustrated in FIG. 4, the distal end part 36a of the second catheter body 36 includes a second inner surface portion 42 and a second outer surface portion 44. The second inner surface portion 42 is a part of the second lumen 34 and has a reverse tapered shape expanding in diameter in the distal direction (direction of arrow B). In other words, the second inner surface portion 42 has a tapered shape in which the diameter gradually decreases from a most distal end 36b of the second catheter body 36 toward the proximal end.

The distal end part 36a of the second catheter body 36 is an end portion in an advancing direction (direction of arrow B) when the retrograde catheter 20 is inserted into the blood vessel 14 and moved forward. The second inner surface portion 42 is disposed in a predetermined range from the most distal end 36b of the second catheter body 36 toward the proximal end.

The second outer surface portion 44 is disposed on the outer peripheral surface of the second catheter body 36. The second outer surface portion 44 has a tapered shape whose diameter decreases in the distal direction (direction of arrow B) of the second catheter body 36. The second outer surface portion 44 is disposed in a predetermined range from the most distal end 36b of the second catheter body 36 toward the proximal end. That is, the distal end part 36a of the second catheter body 36 gradually tapers toward the most distal end 36b (distal direction, direction of arrow B).

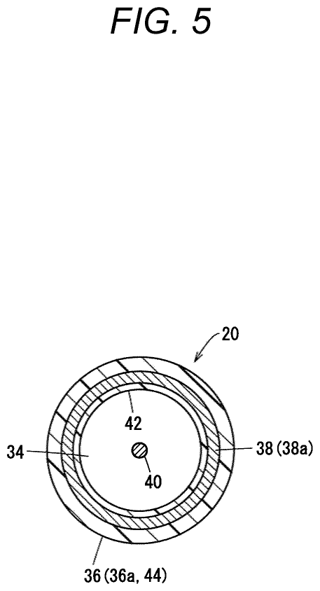

The second marker 38 is formed in a tubular shape from a radiopaque material (for example, gold, platinum, tungsten, a mixture thereof, or the like) (see FIG. 5). The second marker 38 enables the distal position of the retrograde catheter 20 to be visually recognized in the living body 12 under angiography.

The second marker 38 is embedded in the distal end part 36a of the second catheter body 36. The second marker 38 is formed in a reverse tapered shape expanding in diameter toward the distal direction of the second catheter body 36. The second marker 38 is disposed along the second inner surface portion 42 of the second catheter body 36. The second marker 38 and the second inner surface portion 42 of the second catheter body 36 are substantially parallel to each other. A part of the second marker 38 may be exposed to the second inner surface portion 42. Since the second marker 38 has a reverse tapered shape, the second marker 38 is prevented from falling off from the distal end part 36a of the second catheter body 36 in the distal direction (direction of arrow B).

A distal end part 38a of the second marker 38 has a most distal end 38b disposed in the most distal direction (direction of arrow B). The most distal end 38b of the second marker 38 is disposed on the proximal side (direction of arrow A) of the most distal end 36b of the second catheter body 36. In the axial direction (directions of arrows A and B) of the second catheter body 36, an axial distance L2 between the most distal end 38b of the second marker 38 and the most distal end 36b of the second catheter body 36 is 0.5 mm or less. That is, the most distal end 38b of the second marker 38 is disposed within 0.5 mm on the proximal side (direction of arrow A) from the most distal end 36b of the second catheter body 36. The most distal end 38b of the second marker 38 is not exposed to the outside from the most distal end 36b of the second catheter body 36. The axial length of the second marker 38 is, for example, about 0.5 mm to 1.0 mm along the extending direction of the second catheter body 36.

As illustrated in FIG. 1, the proximal end of the second catheter body 36 includes a second hub 46. The second hub 46 has a tubular shape. The proximal end of the second hub 46 is open. The guide wire 40 can be inserted into the second lumen 34 through the second hub 46.

Next, a case of performing lower limb blood vessel treatment using the catheter system 10 will be described. FIG. 1 is a schematic cross-sectional view illustrating a peripheral portion of the CTO 16a (lesion 16) in a case where the CTO 16a occurs in the blood vessel 14 of a lower limb.

In FIG. 1, the CTO 16a is generated in the blood vessel 14 along the extending direction of the blood vessel 14, and the left side of the CTO 16a is the central side (heart side) and the upstream side of the blood flow. The right side of the CTO 16a is the peripheral side (ankle side) and the downstream side of the blood flow. Hereinafter, the blood vessel 14 on the upstream side (left side) with respect to the CTO 16a is referred to as an upstream blood vessel portion 14a, and the blood vessel 14 on the downstream side (right side) with respect to the CTO 16a is referred to as a downstream blood vessel portion 14b. The upstream blood vessel portion 14a is an artery having a relatively large diameter (inner diameter) of the blood vessel 14. The downstream blood vessel portion 14b is a peripheral blood vessel having a smaller diameter (inner diameter) of the blood vessel 14 than the upstream blood vessel portion 14a.

The diameter (outer diameter) of the antegrade catheter 18 is a diameter suitable for the diameter of the upstream blood vessel portion 14a. The diameter (outer diameter) of the retrograde catheter 20 is a diameter suitable for the diameter of the downstream blood vessel portion 14b. That is, the diameter (outer diameter) of the antegrade catheter 18 is greater than the diameter (outer diameter) of the retrograde catheter 20.

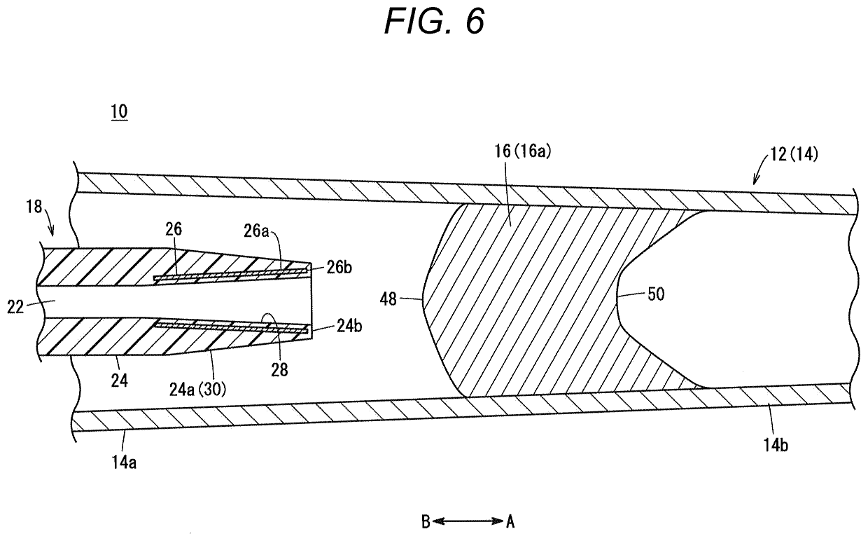

First, as illustrated in FIG. 6, the antegrade approach of the antegrade catheter 18 with respect to the blood vessel 14 of the lower limb of the living body 12 is performed. A medical worker (not illustrated) percutaneously inserts the distal end part 24a of the antegrade catheter 18 into the upstream blood vessel portion 14a of the blood vessel 14. The distal end part 24a of the antegrade catheter 18 is moved forward to the CTO 16a in the peripheral side (direction of arrow A) along a guide wire (not illustrated) in the blood vessel 14. At this time, a medical worker (not illustrated) can perform a procedure while visually recognizing the distal position (most distal end 24b) of the antegrade catheter 18 by visually recognizing the first marker 26 through a display or the like under X-ray contrast effect. With the tubular first marker 26, the first marker 26 can be visually recognized when the distal end part 24a of the antegrade catheter 18 is viewed from any position in the circumferential direction (see FIG. 3).

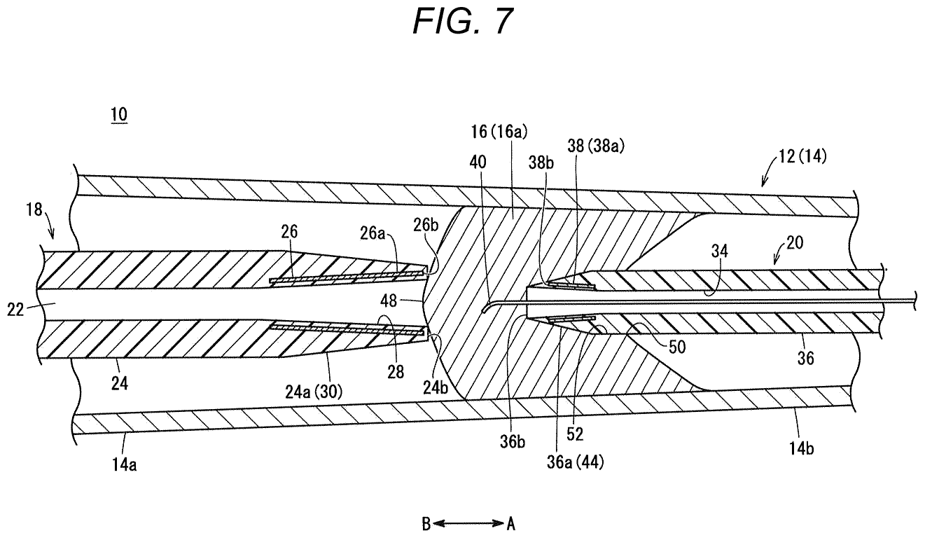

As illustrated in FIG. 7, along the upstream blood vessel portion 14a, the distal end part 24a (most distal end 24b) of the antegrade catheter 18 is delivered to the upstream end portion of the CTO 16a. The upstream end portion of the CTO 16a has a protruding portion 48 protruding toward the upstream blood vessel portion 14a (direction of arrow B). The most distal end 24b of the antegrade catheter 18 contacts the protruding portion 48. A medical worker (not illustrated) can confirm the position of the distal end part 24a of the antegrade catheter 18 by the first marker 26 on a display or the like.

After the distal end part 24a of the antegrade catheter 18 is delivered to the upstream end portion of the CTO 16a, a retrograde approach is performed to deliver the distal end part 36a of the retrograde catheter 20 along the downstream blood vessel portion 14b to the CTO 16a.

As illustrated in FIG. 1, the distal end part 36a of the retrograde catheter 20 is percutaneously inserted into the downstream blood vessel portion 14b of the blood vessel 14. At this time, the guide wire 40 is inserted through the second lumen 34 of the retrograde catheter 20. With the distal end of the guide wire 40 protruding in the distal direction (direction of arrow B) from the most distal end 36b of the retrograde catheter 20 (the distal end of the guide wire 40 is located adjacent the CTO 16a), the distal end part 36a of the retrograde catheter 20 is moved forward to the central side (direction of arrow B) toward the CTO 16a along the guide wire 40. The advancing direction (first direction, toward peripheral side) of the antegrade catheter 18 and the advancing direction (second direction, toward central side) of the retrograde catheter 20 are opposite directions. In other words, the distal end part 36a of the retrograde catheter 20 moves forward to the distal end part 24a of the antegrade catheter 18.

Along the downstream blood vessel portion 14b, the distal end part 36a of the retrograde catheter 20 is delivered to the downstream end portion of the CTO 16a. The downstream end portion of the CTO 16a has a recessed portion 50 recessed toward the upstream blood vessel portion 14a. The most distal end 36b of the retrograde catheter 20 is inserted into the recessed portion 50 and contacts the bottom of the recessed portion 50. The bottom portion is on the most upstream blood vessel portion 14a side in the recessed portion 50.

As illustrated in FIG. 7, by further moving the distal end part 36a of the retrograde catheter 20 forward, the distal end part 36a moves forward from the recessed portion 50 to the inside of the CTO 16a. The distal end part 36a (most distal end 36b) of the retrograde catheter 20 digs (digs into or penetrates) the CTO 16a to form a perforation 52. The perforation 52 is formed from the bottom of the recessed portion 50 toward the protruding portion 48. As the retrograde catheter 20 progresses, the perforation 52 penetrates to the upstream end portion (protruding portion 48) of the CTO 16a.

Since the upstream end portion of the CTO 16a is convex (protruding portion 48) toward the central side, it is difficult to excavate (dig out, cut out or penetrate into) the CTO 16a toward the peripheral side by the antegrade catheter 18. On the other hand, since the downstream end portion of the CTO 16a is recessed (recessed portion 50) toward the central side, it is easy to excavate (dig out, cut out or penetrate into) the CTO 16a toward the central side by the retrograde catheter 20, and it is easy to move forward to the CTO 16a or the vicinity of the center in the blood vessel 14.

A medical worker (not illustrated) checks the relative position between the first marker 26 of the antegrade catheter 18 and the second marker 38 of the retrograde catheter 20 under angiograhpy, and aligns the first marker 26 and the second marker 38 so as to be arranged on a straight line along the extending direction (direction of arrow A, B) of the blood vessel 14. The straight line is a straight line that at least partially located in the second lumen 34 of the tubular second catheter body 36 of the retrograde catheter 20 and the first lumen 22 of the distal end part 24a of the antegrade catheter 18 as generally shown in FIG. 7. By aligning the first marker 26 and the second marker 38, the most distal end 24b of the antegrade catheter 18 and the most distal end 36b of the retrograde catheter 20 are arranged on a straight line at the CTO 16a.

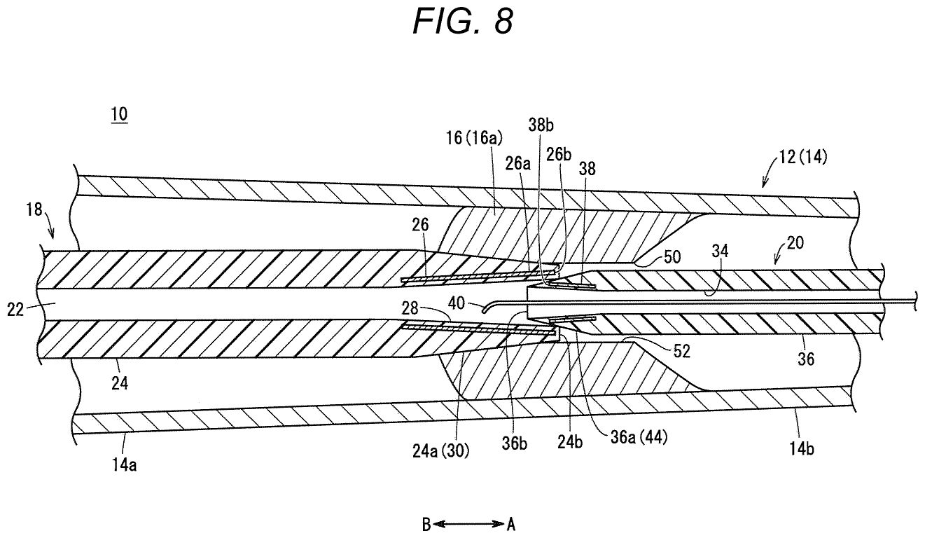

As illustrated in FIG. 8, by bringing the distal end part 24a of the antegrade catheter 18 and the distal end part 36a of the retrograde catheter 20 close to each other, the distal end part 24a (most distal end 24b) of the antegrade catheter 18 moves forward into the CTO 16a along the perforation 52. Inside the CTO 16a, the distal end part 36a of the retrograde catheter 20 is inserted into the first lumen 22 of the distal end part 24a of the antegrade catheter 18. At this time, since the perforation 52 is formed in the CTO 16a, the distal end part 24a of the antegrade catheter 18 is smoothly inserted into the CTO 16a along the perforation 52. The perforation 52 is expanded radially outward by the distal end part 24a of the antegrade catheter 18. The first outer surface portion 30 provides good insertability of the antegrade catheter 18 into the perforation 52.

After the distal end part 24a of the antegrade catheter 18 is placed at a predetermined position inside the CTO 16a, the retrograde catheter 20 is removed. The balloon catheter (not shown) is inserted through the first lumen 22 of the antegrade catheter 18, and the balloon catheter is delivered to the CTO 16a. The balloon catheter treats the CTO 16a. After an antegrade guide wire (not illustrated) is inserted through the first lumen 22 of the antegrade catheter 18, the antegrade catheter 18 may be removed to indwell the antegrade guide wire, and the balloon catheter may be delivered to the CTO 16a along the antegrade guide wire (not illustrated).

The insertion of the distal end part 36a of the retrograde catheter 20 into the distal end part 24a of the antegrade catheter 18 is not limited to the case of being performed inside the CTO 16a. In the upstream blood vessel portion 14a or the downstream blood vessel portion 14b in the vicinity of the CTO 16a, the distal end part 36a of the retrograde catheter 20 may be inserted into the distal end part 24a of the antegrade catheter 18. By inserting only the guide wire 40 into the distal end part 24a of the antegrade catheter 18 and arranging the guide wire 40, the first marker 26, and the second marker 38 coaxially, if the retrograde catheter 20 is easily pushed and the perforation 52 penetrates, it is not necessary to move forward to the position where the retrograde catheter 20 is inserted into the distal end part 24a of the antegrade catheter 18.

As described above, in the embodiment disclosed here as an example of the new catheter and catheter system, and operational procedure using such catheter and catheter system, since the axial distance L1 between the most distal end 26b of the first marker 26 disposed at the distal end part 24a of the antegrade catheter 18 and the most distal end 24b of the first catheter body 24 is 0.5 mm or less, when the antegrade catheter 18 is moved forward to the peripheral side (direction of arrow A) along the blood vessel 14, the distal position (most distal end 24b) of the antegrade catheter 18 can be accurately confirmed under angiography. Since the axial distance L2 between the most distal end 38b of the second marker 38 disposed at the distal end part 36a of the retrograde catheter 20 and the most distal end 36b of the second catheter body 36 is 0.5 mm or less, when the retrograde catheter 20 is moved forward to the central side (direction of arrow B) along the blood vessel 14, the distal position (most distal end 36b) of the retrograde catheter 20 can be accurately confirmed under angiography. Since the first and second markers 26 and 38 are formed in the reverse tapered shape, the first and second markers 26 and 38 can be easily identified based on the shape that can be confirmed under X-ray contrast effect, and the first and second markers 26 and 38 are suitably prevented from falling off from the first and second catheter bodies 24 and 36.

By disposing the first marker 26 at the distal end part 24a of the antegrade catheter 18 and disposing the second marker 38 at the distal end part 36a of the retrograde catheter 20, it is easy to align the distal end part 24a (most distal end 24b) of the antegrade catheter 18 and the distal end part 36a (most distal end 36b) of the retrograde catheter 20.

In lower limb blood vessel treatment, when the distal end part 36a of the retrograde catheter 20 is inserted into the first lumen 22 of the distal end part 24a of the antegrade catheter 18, the distal end part 36a of the retrograde catheter 20 can be smoothly inserted into the first lumen 22 by the first inner surface portion 28 of the first catheter body 24. As a result, it is possible to enhance the insertability between the distal end part 24a of the antegrade catheter 18 and the distal end part 36a of the retrograde catheter 20.

In lower limb blood vessel treatment, when the distal end part 36a of the retrograde catheter 20 is inserted into the first lumen 22 of the distal end part 24a of the antegrade catheter 18, the second outer surface portion 44 of the retrograde catheter 20 can enhance the insertability of the retrograde catheter 20 with respect to the first lumen 22.

The present invention is not limited to the above disclosure, and various configurations can be adopted without departing from the gist of the present invention. For example, the lesion 16 may not be the representative CTO 16a, but may be a stenosis lesion with an intensity at which blood flow is recognized to some extent, or may be a mild stenosis lesion in order to reduce the burden on the patient.

The detailed description above describes embodiments of a catheter, catheter system and operational method representing examples of the new catheter, catheter system and operational procedure or method of use disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A catheter that is insertable into a lumen of a living body and movable forward along the lumen, the catheter comprising:
a tubular catheter body having a proximal end part and a distal end part at opposite ends of the tubular catheter body, the tubular catheter body also having a lumen that extends throughout the tubular catheter body in a distal direction from the proximal end part of the tubular catheter body to the distal end part of the tubular catheter body;
a tubular marker comprised of a radiopaque material and disposed at the distal end part of the tubular catheter body;
the tubular marker being having a reverse tapered shape in which the tubular marker expands in outer diameter toward the distal direction of the catheter body; and
a distance between a most distal end of the tubular marker and a most distal end of the tubular catheter body in an axial direction of the catheter body is 0.5 mm or less.

2. The catheter according to claim 1, wherein the catheter is: i) an antegrade catheter that is movable forward toward a peripheral side of the living body along the lumen; or ii) a retrograde catheter that is movable forward toward a central side of the living body along the lumen and has a distal end insertable into a distal end of the antegrade catheter.

3. The catheter according to claim 1, wherein the catheter is an antegrade catheter that is movable forward to a peripheral side of the living body along the lumen.

4. The catheter according to claim 3, wherein
the distal end part of the tubular catheter body has an inner surface portion that is a part of the lumen, the inner surface portion having a reverse tapered shape expanding in diameter in the distal direction, and
the marker has an inner surface that is spaced radially outwardly from the inner surface portion of the tubular catheter body.

5. The catheter according to claim 1, wherein
the catheter is a retrograde catheter that is movable forward to a central side of the living body along the lumen,
a distal end of the retrograde catheter is insertable into the lumen at a distal end of an antegrade catheter that is movable forward to a peripheral side of the living body along the lumen, and
an outer peripheral surface of the distal end of the retrograde catheter has a tapered outer surface portion whose outer diameter decreases in the distal direction.

6. The catheter according to claim 1, wherein the distal end part of the tubular catheter body has an inner surface surrounding the lumen in the tubular catheter body, the marker having an inner surface that faces toward the lumen, the inner surface of the marker and the inner surface of the distal end part of the tubular catheter body being substantially parallel to each other.

7. The catheter according to claim 1, wherein the distal end part of the tubular catheter body at which the marker is located has an outer surface that tapers in a narrowing manner toward the most distal end of the tubular catheter body so that an outer diameter of the distal end part of the tubular catheter body at which the marker is located decreases toward the most distal end of the tubular catheter body.

8. The catheter according to claim 1, wherein the marker has oppositely facing inner and outer surfaces that are both completely covered.

9. The catheter according to claim 1, wherein the lumen in the distal end part of the tubular catheter body has an inner diameter that increases in the distal direction so that the inner diameter of the lumen is greatest at the most distal end of the tubular catheter body.

10. A catheter system that is insertable into a lumen of a living body and is movable along the lumen, the catheter system comprising:
an antegrade catheter that is movable forward to a peripheral side of the living body along the lumen;
a retrograde catheter that is movable forward to a central side of the living body along the lumen;
the antegrade catheter including:
a tubular first catheter body having a first lumen;
a tubular first marker comprised of a radiopaque material and disposed at a distal end part of the tubular first catheter body;
the tubular first marker having a reverse tapered shape in which an outer diameter of the tubular first marker expands in a distal direction of the tubular first catheter body;
a distance between a most distal end of the tubular first marker and a most distal end of the tubular first catheter body in an axial direction of the tubular first catheter body is 0.5 mm or less,
a distal end of the retrograde catheter is insertable into a distal end of the antegrade catheter and into the first lumen;
the retrograde catheter includes:
a tubular second catheter body having a second lumen;
a tubular second marker comprised of a radiopaque material and disposed at a distal end part of the tubular second catheter body;
the tubular second marker has a reverse tapered shape in which an outer diameter of the tubular second marker expands in a distal direction of the tubular second catheter body;
a most distal end of the tubular second marker is disposed on a proximal side with respect to a most distal end of the tubular second catheter body; and
a distance between the most distal end of the tubular second marker and the most distal end of the tubular second catheter body in an axial direction of the tubular second catheter body is 0.5 mm or less.

11. The catheter system according to claim 10, wherein the distal end part of the tubular second catheter body has an inner surface surrounding the second lumen in the tubular second catheter body, the tubular second marker having an inner surface that faces toward the second lumen, the inner surface of the tubular second marker and the inner surface of the distal end part of the tubular second catheter body being substantially parallel to each other.

12. The catheter system according to claim 11, wherein the distal end part of the tubular first catheter body has an inner surface surrounding the first lumen in the tubular first catheter body, the tubular first marker having an inner surface that faces toward the first lumen, the inner surface of the tubular first marker and the inner surface of the distal end part of the tubular first catheter body being substantially parallel to each other.

13. The catheter system according to claim 10, wherein the distal end part of the tubular second catheter body at which the second marker is located has an outer surface that tapers in a narrowing manner toward the most distal end of the tubular second catheter body so that an outer diameter of the distal end part of the tubular second catheter body at which the second marker is located decreases toward the most distal end of the tubular second catheter body.

14. The catheter system according to claim 13, wherein the distal end part of the tubular first catheter body at which the first marker is located has an outer surface that tapers in a narrowing manner toward the most distal end of the tubular first catheter body so that an outer diameter of the distal end part of the tubular first catheter body at which the first marker is located decreases toward the most distal end of the tubular first catheter body.

15. The catheter system according to claim 10, wherein the tubular second marker has oppositely facing inner and outer surfaces that are both completely covered.

16. The catheter system according to claim 10, wherein the second lumen in the distal end part of the tubular second catheter body has an inner diameter that increases in the distal direction so that the inner diameter of the second lumen is greatest at the most distal end of the tubular second catheter body.

17. The catheter system according to claim 10, wherein
the distal end part of the tubular second catheter body has an inner surface surrounding the second lumen, the inner surface of the distal end part of the tubular second catheter body portion having a reverse tapered shape that expands in diameter in the distal direction, and
the second marker has an inner surface that is spaced radially outwardly from the inner surface portion of the tubular second catheter body.

18. The catheter system according to claim 17, wherein
the distal end part of the tubular first catheter body has an inner surface surrounding the first lumen, the inner surface of the distal end part of the tubular first catheter body portion having a reverse tapered shape that expands in diameter in the distal direction, and
the first marker has an inner surface that is spaced radially outwardly from the inner surface portion of the tubular first catheter body.

19. A treatment method for treating a lesion in a lumen of a living body using an antegrade catheter and a retrograde catheter,
the antegrade catheter including:
a tubular first catheter body having a first lumen;
a tubular first marker comprised of a radiopaque material and disposed at a distal end part of the tubular first catheter body;
the tubular first marker having a reverse tapered shape in which an outer diameter of the tubular first marker expands in a distal direction of the tubular first catheter body;
a distance between a most distal end of the tubular first marker and a most distal end of the tubular first catheter body in an axial direction of the tubular first catheter body is 0.5 mm or less;
the retrograde catheter including:
a tubular second catheter body having a second lumen;
a tubular second marker comprised of a radiopaque material and disposed at a distal end part of the second catheter body;
the tubular second marker has a reverse tapered shape in which an outer diameter of the tubular second marker expands in a distal direction of the second catheter body;
a most distal end of the tubular second marker is disposed on a proximal side with respect to a most distal end of the tubular second catheter body;
a distance between the most distal end of the tubular second marker and the most distal end of the tubular second catheter body in an axial direction of the tubular second catheter body is 0.5 mm or less;
the method comprising:
moving the antegrade catheter forward along the lumen of the living body toward the lesion from a position upstream of the lesion considered with reference to a blood flow direction;
moving the retrograde catheter forward along the lumen of the living body toward the lesion from a position downstream of the lesion considered with reference to the blood flow direction;
aligning the tubular first marker and the tubular second marker while confirming positions of the tubular first marker and the tubular second marker under angiography so that the distal end part of the tubular first catheter body of the antegrade catheter and the distal end part of the tubular second catheter body of the retrograde catheter are aligned; and
inserting a distal end of the retrograde catheter into a distal end of the antegrade catheter.

20. The method according to claim 19, further comprising forming a perforation in a part of the lesion facing downstream of the lesion by moving the retrograde catheter so that the distal end of the retrograde catheter digs into the lesion.
